(19) United States
(12) Patent Application Publication
(10) Pub. No.: US 2003/0180358 A1
(43) Pub. Date: Sep. 25, 2003

(54) System and method for affecting the pH state of the contents of the distal bowel in mammals

(76) Inventor: Walter William Stumberger, Hightstown, NJ (US)

Correspondence Address: Walter William Stumberger, 203 Dutch Neck Road, Hightstown, NJ, 08520, US

(21) Appl. No.: 10/105,712

(22) Filed: Mar. 25, 2002

(51) Int. Cl. A61K 9/22; A61K 33/00

(52) U.S. Cl. 424/468; 424/600

(57) ABSTRACT

This invention is a system and method for affecting the pH state of the contents of the distal bowel while not significantly impacting the chemistry of the contents of the proximal bowel. This is accomplished by controlling the release of pH-affecting agents until they reach a selected physiological region or digestive stage beyond the stomach.

### STATEMENT REGARDING FEDERALLY SPONSORED RESEARCH OR DEVELOPMENT

No federal funds were used to sponsor the research or development of this invention.

### BACKGROUND OF THE INVENTION

Under certain circumstances, the ability to adjust the pH (Alkaline/Acid) ratio of the contents of the distal bowel in mammals without significantly affecting the natural chemistry of the proximal digestive system is important.

Current medical technology adjusts the pH ratio of the contents of the distal bowel by adjusting the pH ratio of the entire contents of the digestive system usually through the use of antacid (acid neutralizing) chemicals ingested by mouth or placed in the stomach by other means. This method however affects the entire digestive process and can lead to mal-absorption or an inability to turn food into useable nutrients inside the digestive system during the critical first half of the digestive process.

This invention addresses the limitation of the current medical technology by presenting a novel method and system for controlling the release of the pH-affecting agents taken by mouth or otherwise placed in the stomach until the pH-affecting agents are further along the physiology and chemistry of the digestive process.

No medical claims are made.

### BRIEF SUMMARY OF THE INVENTION

This invention is a method and system for affecting the pH of the contents of the distal bowel through the use of an arrangement to delay or control the release of an orally ingested pH-affecting chemical, a pH-affecting compound, a plurality of pH-affecting chemicals, or a plurality of pH-affecting compounds, until the chemicals or compounds have passed through the stomach and reached a selected physiological region or digestive stage.

In one common and primitive embodiment this invention can be an orally ingested delay-released antacid.

From the foregoing it will be appreciated by those skilled in the art that the present invention provides a particularly effective and advantageous method of overcoming many of the limitations associated with affecting the pH of the distal digestive system by releasing a pH-affecting chemical or compound, or plurality of pH-affecting chemicals or compounds within the region of the proximal digestive system. It will also be readily appreciated by one with ordinary skill in the art to use the method and system of the presented invention in other configurations and that these other configurations are part of this invention.

### BRIEF DESCRIPTION OF THE SEVERAL VIEWS OF THE DRAWING

FIG. 1 is a schematic diagram of the invention demonstrating the concept of placing a pH-affecting chemical a pH-affecting compound or a plurality of pH-affecting chemicals or pH-affecting compounds (1) inside a release controlling agent or system (2).

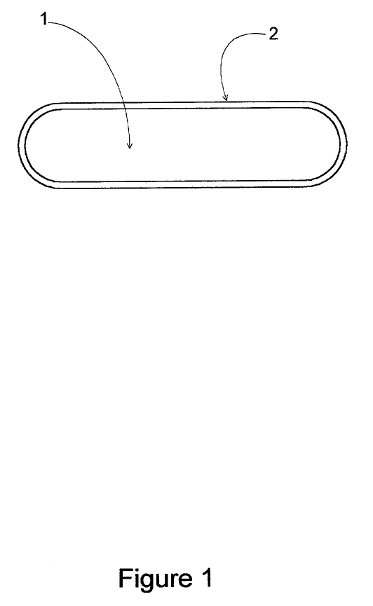

FIG. 2 is a schematic diagram of the invention demonstrating the concept of the invention which includes the concept of placing multiple pH-affecting chemicals or multiple pH-affecting compounds or a multiple plurality of pH-affecting chemicals or a multiple plurality of pH-affecting compounds (1, 3, 4, and 5) inside a release controlling agent or system (2).

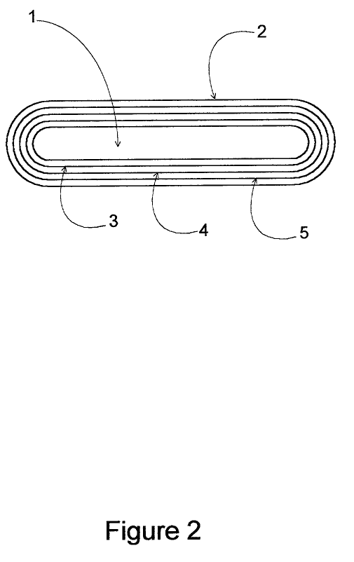

This invention is not shape or size dependent.

Although certain presently preferred embodiments of the present invention have been specifically described herein it will be apparent to those skilled in the art to which the invention pertains that variations and modifications of the various embodiments shown and described herein may be made without departing from the spirit and scope of the invention.

### DETAILED DESCRIPTION OF THE INVENTION

The invention is a method and system for affecting the pH state of the distal bowel contents and distal digestive system through the controlled release of a pH-affecting chemical or pH-affecting compound or a plurality of pH-affecting chemicals or pH-affecting compounds. With said controlled release being the function of a chemical or compound or plurality of chemicals and compounds used to control the release of the said pH-affecting chemical the said pH-affecting compound the said plurality of pH-affecting chemicals and/or the said plurality pH-affecting compounds until they reach a distal or otherwise selected physiological region or digestive stage.

While this illustrates and describes what are at present considered to be preferred embodiments of the present invention it will be understood by those skilled in the art that various changes and modifications may be made, and equivalents may be substituted for elements thereof without departing from the true scope of the present invention. In addition, many modifications may be made to adapt a particular situation or material to the teaching of this invention without departing from the central scope of this invention. Therefore, it is intended that the present invention not be limited to the particular embodiment disclosed as the best mode contemplated for carrying out the present invention, but that the present invention includes all embodiments falling within the scope of the appended claims.

1. I claim that this invention is a system and method for affecting the pH state of the distal bowel contents,
with said pH adjustment of the distal bowel contents being the function of a pH-affecting chemical or pH-affecting compound or a plurality of pH-affecting chemicals or plurality of pH-affecting compounds, and
with the release into the distal bowel of said pH-affecting agents being controlled by a chemical or compound or plurality of chemicals or compounds providing a controlling system, and
with said controlling system providing a delay in the release of the said pH-affecting agent until the said pH-affecting agent reaches a desired physiological region or selected digestive stage within the bowel.

2. I claim that this invention is a system and method for affecting the pH state of the distal bowel contents,
with said pH adjustment of the distal bowel contents being the function of a plurality of pH-affecting chemicals or a plurality of pH-affecting compounds or a multiple plurality of pH-affecting chemicals or a multiple plurality of pH-affecting compounds, and
with the release into the distal bowel of the said pH-affecting agents being controlled by a chemical or compound or plurality of chemicals or compounds providing a controlling system and
with said controlling system providing a delay in the release of the said pH-affecting agents until the said pH-affecting agents reach a desired physiological region or selected digestive stage within the bowel.
